(19) United States
(12) Patent Application Publication
(10) Pub. No.: US 2007/0265185 A1
(43) Pub. Date: Nov. 15, 2007

(54) Phosphorylated Vimentin Serving as a Marker of the Aggressiveness and/or Invasiveness of Tumors

(75) Inventors: Ali Bouamrani, Grenoble (FR); Emmamuel Gay, Meylan (FR); Jean-Paul Issartel, Saint Egreve (FR); Francois Berger, Meylan (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Genome Express SA, Paris (FR)

(21) Appl. No.: 11/663,127

(22) PCT Filed: Sep. 15, 2005
(86) PCT No.: PCT/EP05/54598

§ 371 Date: Jun. 28, 2007

(30) Foreign Application Priority Data
Sep. 17, 2004 (FR) 0409857

(51) Int. Cl. A61K 31/00; A61P 43/00; G01N 33/574

(52) U.S. Cl. 514/1; 435/7.23

(57) ABSTRACT

The invention relates to a novel method used for deter-mining the aggressiveness and/or invasiveness of a tumor. The invention also relates to a method for identifying an anticancer agent that makes it possible to reduce the aggressiveness and/or invasiveness of a tumor. The invention is based on the inventors' findings that vimentin produced by the cancer cells can be a marker of the aggressiveness and/or invasiveness of a tumor. This maker capacity is not associated with expression of the vimentin in the cancer cells but is with a post-translational transformation of this different protein according to the degree of aggressiveness and/or invasiveness.

The present invention relates to a novel method that can be used for determining the aggressiveness and/or invasiveness of a tumor, and also to a method for identifying an anticancer agent for reducing the aggressiveness and/or invasiveness of a tumor.

The invention is based on the inventors' findings that the vimentin produced by cancer cells could be a marker for the aggressiveness and/or invasiveness of a tumor. This marker quality was not associated with the level of expression of vimentin in cancer cells, but with a post-translational transformation, of this protein, which is different according to the degree of aggressiveness and/or invasiveness.

Phosphorylated vimentin can be specifically detected in noninvasive tumors. Conversely, this marker is present in a significantly much lower amount in tumors with a poor prognosis, i.e. tumors whose invasive nature threatens the survival of patients presenting tumors of this type.

Neuropathological diagnosis of tumors combines the evaluation of two fundamental parameters (Chatel and Brucher, 2001; Daumas-Duport and Figarella-Branger, 2002):
A cytological criterion, which corresponds to the cell typing. This typing consists in determining the histological origin of the tumor. Astrocytomas (astrocyte cell tumors), oligodendrogliomas (oligodendrocyte tumors), meningiomers (tumors of the meninges) are, for example, distinguished; and also many other types of tumors.
A histoprognostic criterion, which, according to microscopic observational elements of the morphology of the cells, consists in assigning to the tumor a malignancy grade. Tumors can be categorized according to a scale of a maximum of four grades (according to the classifications in force that are used), which predict the degree of progression and of invasiveness of the tumor lesion. Certain tumors are relatively nonprogressive, others grow very rapidly.

At the current time, this histological analysis carried out by the anatomy-pathologist is predominant, and unfortunately the diagnostic disagreements seen between experts in the field are enormous (up to 64% disagreement according to the tumors). Worse still, similar disagreements can be noted when interpretations of identical samples are entrusted to the same person a few weeks apart. This observation is worrying given that diagnostic errors can lead to needless radiotherapy and/or chemotherapy that has serious consequences for the patient.

To supplement the histological analysis, only a few rare diagnostic tests based on molecular approaches exist. Cytological observations can thus be supplemented with the search for genetic anomalies and, in some laboratories, the detection of certain protein markers using specific antibodies.

By way of examples, mention may be made of:
for cytogenetic analyses: the search of chromosomal anomalies such as deletions or amplifications of regions, heterozygocity losses (in particular allelic losses in 1p and 19q for oligodendrogliomas (Cairncross et al., 1998)), searches for markers that signal chromosomal fragmentations and translocations. These events, which can affect various chromosomal regions of several chromosomes, are often detected using methods such as: karyotype definition, Comparative Genomic Hybridization (CGH), in situ hybridization of fluorescent probes (FISH), or by PCR-based techniques;
for the search for protein markers: immunohisto-chemical detection of glyofibrillary acidic protein (GFAP) which is an indicator of normal or tumoral astrocytes; of the Ki 67 proliferation antigen.

For the moment, no routine tests based on detection and quantification of transcriptomic tumor marker concentration exists.

The few molecular tests currently available do not make it possible to distinguish unambiguously the various tumor cell types, and especially do not make it possible to give a correct prognosis of their aggressiveness and invasiveness.

The relevance of the diagnosis has a predominant effect on the therapeutic action undertaken by the clinician, who has a panoply of means for combating tumors.

Exeresis of the tumor constitutes one means, a priori radical, for eradicating a tumor. In clinical practice, this strategy remains imperfect. The tumor ablation must be exhausted, which is far from being the case, either because the limits of the tumor remain difficult to evaluate at the time of diagnosis, or because tumors that are infiltrating in nature have already “radiated” pretumor cells into the brain regions neighboring the primary tumor. Finally, surgery constitutes an essentially traumatic procedure, and the operability of tumors must always be evaluated critically according to the benefit-risk to the patient assessment.

It is therefore important to be able to have new methods for characterizing the aggressiveness and/or invasiveness of the tumor.

Vimentin is a 465 amino acid protein of molecular mass 53554 Da (Swiss-Prot (http://www.expasy.org) accession number: P08670).

The gene encoding vimentin is located on chromosome 10 in region 10p13 (Ferrari et al., 1987). A single copy of this gene exists in the human haploid genome (a pseudogene has been identified on chromosome 6; cyto-genetic region 6q22.32). A single transcript of approximately 1.8 kbp is usually detected in cells expressing vimentin (Lilienbaum et al., 1986; Perreau et al., 1988). Several polymorphisms or sequencing conflicts are reported in the literature (Ferrari et al., 1986; Perreau et al., 1988; Honore et al., 1990).

According to immunohistochemical examinations, vimentin, just like GFAP, is found in neither medulloblastomas nor oligodendrogliomas (Yung et al., 1985), but is clearly present in all astrocytomes (Yang et al., 1994).

Numerous data in the literature suggest that, in epithelial cancer cells, there is an aberrant expression of vimentin. This phenomenon of intermediate filament protein expression in epithelial cells, with the acquisition of migratory and/or invasive properties, is called epithelial-mesenchymal transition (EMT).

Vimentin is detected in many hormone-independent breast carcinomas (epithelial tumors) (Cattoretti et al., 1988; Sommers et al., 1989). Hyperexpression of vimentin is thought to correlate with a marked invasive and metastatic nature in the case of cervical carcinoma (Gilles et al., 1996). The detection of vimentin in kidney carcinoma cells is a marker of poor prognosis (Donhuijsen and Schulz, 1989); in these tumors, there is thought to be a relationship between vimentin expression, nuclear DNA content and histological grade (Dierick et al., 1991). For certain prostate tumor cell lines, motility but not invasiveness can be correlated with a greater expression of vimentin in vitro. These tumors which express vimentin are capable of producing bone metastases (Lang et al., 2002). Hep3B hepato-carcinoma cells treated with phorbol esters (activators of protein kinases C, PKCs) or with retinoic acid show respectively an increase in the level of vimentin transcript or a decrease. These compounds do not affect cell proliferation, but the invasive potential, measured in vitro, is increased by phorbol esters and reduced with retinoic acid (Yoon et al., 2004). In addition, the coexpression of vimentin and keratins in melanoma tumor cells or breast cancers is judged by several authors to be a factor that increases the proliferative and metastatic capacities of these tumors (Chu et al., 1996, Hendrix et al., 1997).

According to the publications mentioned above, it appears to be credible to conclude that the invasiveness of tumor cells will be directly proportional to the cellular concentration of vimentin. However, other studies which do not confirm this message are also found. In breast tumors, it has been indicated that vimentin expression is an element which should not be associated with an increased risk of death or be considered as a factor for poor prognosis (Seshadri et al., 1996). For other authors, the measurement of vimentin expression level is not a parameter that makes it possible to give a diagnosis or to predict tumor progression capacities (Raymond and Leong, 1989; Holck et al., 1993; Heatley et al., 1995, 2002).

To put it plainly, the level of expression of vimentin does not necessarily correlate with the invasiveness, depending on the cell lines tested. The transcriptional control of vimentin expression does not therefore appear to be the only criterion for rationalizing tumor invasiveness.

Vimentin can exist in a nonphosphorylated form and multiple phosphorylated forms. Vimentin phosphorylation plays an important role in the disassembly of intermediate filaments. Several serine residues predominantly located in the N-terminal portion of the protein constitute the sites for posttranslational modification by phosphorylation (Ando et al., 1989, 1991; Chou et al., 1991; Huang et al., 1994). Several different kinases can be involved in these vimentin modifications (Tsujimura et al., 1994). In proliferating cells, vimentin has various statuses: phosphorylated or nonphosphorylated according to the various mitotic stages (Tsujimura et al., 1994).

According to the invention, the term “aggressiveness” is intended to mean the characteristic that defines the malignant nature of tumors. Tumors are generally classified according to several grades (the grades are established on an evolving scale, predominantly and according to tumor types, on levels of I to IV). The grade is defined by combining various criteria based on clinical observation, and histological and cytological parameters.

According to the invention, the term “invasiveness” is intended to mean the ability, for a high-grade malignant tumor, to propagate outside the tissue in which it arose. This propagation goes together with a colonization of further histological structures, the tumorization of adjacent tissues and/or the possibility of more or less generalizable invasion of the organism via metastases.

The present invention therefore relates to a method that can be used for determining the aggressiveness and/or invasiveness of a tumor, said method comprising, on a sample of tumor cells taken beforehand, the steps consisting in:
preparing a biological extract comprising vimentin, and
determining whether the vimentin thus extracted is phosphorylated or nonphosphorylated,
   the significant presence of phosphorylated vimentin being an indication of nonaggressiveness and/or noninvasiveness of the tumor.

For the method according to the invention, it will be possible to establish the average content of phosphorylated vimentin on samples of tumor cells known for their aggressiveness and/or invasiveness, and then subsequently to compare the content of phosphorylated vimentin of a new sample to be analyzed, with this average content, which is a standard value for invasiveness and/or aggressiveness.

It will be possible to determine, in relation to the average content associated with invasive tumors, whether the content of phosphorylated vimentin of the tumor to be analyzed is significantly high—an indication of noninvasiveness and/or nonaggressive-ness—or significantly low—an indication of invasiveness and/or aggressiveness.

For the method according to the invention, it will also be possible to establish the average content of phosphorylated vimentin on samples of tumor cells known for their nonaggressiveness and/or noninvasiveness, and then subsequently to compare the content of phosphorylated vimentin of a new sample to be analyzed, with this average content, which is a standard value for noninvasiveness and/or nonaggressiveness.

It will be possible to determine, in relation to this average content associated with noninvasive and/or nonaggressive tumors, whether the content of phosphorylated vimentin is significantly high or low.

Those skilled in the art may choose to carry out a comparison in relation to just one of the two standard values or in relation to both the standard values above.

According to another embodiment of the invention, it will be possible to determine, on the same example, the content of phosphorylated vimentin and the content of nonphosphorylated vimentin, the comparison of the two values make it possible to determine whether the content of phosphorylated vimentin is significantly high or low.

To implement the method according to the invention, it is determined whether the vimentin is phosphorylated or nonphosphorylated, preferably by means of a method selected from the group comprising:
SELDI-TOF analysis
the FACS method or equivalents thereof
RIA isotopic labeling
FRET fluorescence transfer
the demonstration of immunocomplexes
methods for detecting antigen-antibody reactions, such as ELISA methods, protein microarrays or Western blotting
electrophoretic migration tests with the demonstration of posttranslational modifications of vimentin.

Depending on the method of detection chosen, the detection of phosphorylated or nonphosphorylated vimentin in the biological extract containing it comprises the demonstration of vimentin and/or of vimentin fragments.

Those skilled in the art will be able to determine the vimentin fragments that make it possible to implement the method according to the invention.

According to a first embodiment of the invention, the phosphorylation-modified vimentin is extracted from a sample such as a biopsy and then analyzed and detected by the experimental approach based on mass spectrometry, referred to as SELDI technology (Surface Enhanced Desorption/Ionization technology) (Merchant and Weinberger, 2000; Weinberger et al., 2000). Briefly, this platform allies an enrichment in protein fractions, from a biological sample, on a substrate of activated chemical matrix type, coupled to mass spectrometry analysis of the proteins thus adsorbed onto the substrate. This embodiment is implemented by using the technical platform distributed by the company Ciphergen (Ciphergen Biosystems, Inc. Fremont, Calif., USA; http://www.ciphergen.com). Example 1 illustrates the results obtained for the analysis of various types of invasive or noninvasive tumors (meningeal tumors, central nervous system glial cell tumors (glioblastomas and oligodendrogliomas) and lung tumors).

However, it is also possible to make use of other processes or methods of analysis for constituting an application aimed at the diagnosis or prognosis of the state of invasiveness of tumors, and based, in terms of basic principle, on the detection and/or assaying of the identified marker, which is the subject of the invention, or on any product derived from this marker, and also, firstly, on the precursors of this marker or, secondly, the biological elements that are involved in its synthesis, degradation or posttranslational modification. In addition, the setting up of diagnostic tests may require that the nonmodified vimentin and the phosphorylated vimentin be detected or even quantified. This may be required, for example, for the purpose of performing measurements of ratios of amounts between the nonphosphorylated vimentin forms and the phosphorylated vimentin forms.

According to an embodiment other than the SELDI method, the phosphorylated vimentin can first of all be purified from the extract obtained from the samples according to conventional biochemical methods and then detected according to methods chosen from those mentioned above by way of example. In this case, the extraction of the phosphorylated protein is not based, as in the case of the implementation by the SELDI method, on the affinity-capture of the phosphorylated protein on surfaces of activated material (ProteinChip® type from Ciphergen). After enrichment of the extract in phosphorylated vimentin, or even purification of said protein, the detection of this protein can be obtained according to one of the two approaches described hereinafter, which allow the detection of all, or of fragments corresponding to or derived from, the protein in its nonphosphorylated form:
1. The nonmodified vimentin can be detected as it is after chemical or enzymatic removal of the phosphate groups (dephosphorylation) of the phosphorylated vimentin extracted from the samples. This approach, illustrated in example 2, can be carried out as a supplement to the test for detecting phosphorylated vimentin and would, in this case, make it possible to confirm that phosphorylated vimentin is indeed present in the sample analyzed.
2. Finally, it is also possible to envision detecting the presence or disappearance of peptides derived from enzymatical or chemical cleavage of nonmodified vimentin. An illustration of analytical results obtained by detecting peptides from enzymatic digestion is shown in example 3. In addition, the cleavage of phosphorylated vimentin by chemical or enzymatic reactions that include chemical modifications of phosphorylated amino acids (according to methods based on those described, for example, by Rusnak et al., 2002 or Knight et al., 2003) could result in the creation of vimentin peptides identical to fractions of nonmodified vimentin and which would then be detectable as such according to suitable methods.

Other analytical techniques can also be used for detecting the phosphorylated or nonphosphorylated vimentin form(s), in order to carry out an analytical biological test for diagnostic purposes. The non-exhaustive list described hereinafter is of value as an example of varied techniques that those skilled in the art are able to use in order to carry out an analysis of tumor samples so as to detect all or part of the modified vimentin and to obtain therefrom information of diagnostic or prognostic value in accordance with the invention described herein. The analyses may be carried out directly on crude samples, fragments or tissue sections (biopsies) or on samples that have undergone treatments, corresponding, nonexhaustively, to lysates, extracts or subfractions derived from these tumors. It is also possible to envision searching for the marker or for these derivatives in biological fluids taken from patients.

The general basic principle that can be used consists in providing components (components that will be referred to as reagents) capable of interacting with the vimentin form(s) or derivatives thereof. On the subject of these reagents, mention may of course be made of polyclonal or monoclonal antibodies and immunoreactive fragments thereof, which may or may not be grafted onto, or with other components; particulate elements capable of interacting with the vimentin forms (recombinant phages or bacteria expressing, at their surface, polypeptide regions capable of interaction with haptens or antigens) (Gao et al., 1999; Knappik et al., 2000); or aptamers (chemical molecules of polynucleotide or even polypeptide type capable of establishing noncovalent high-affinity interactions with target molecules) (Ellington and Szostak, 1990; Tuerk and Gold, 1990). These selective reagents make it possible to demonstrate the modified vimentin in an extremely reliable manner and, depending on the approaches, allow quantification of the amount of this protein in the sample.

The use of such selective reagents can be envisioned through varied methods or protocols which are mentioned below.

Analyses Based on Immunohistochemistry Techniques (Kiernan, 1999):

An approach that is sound and relatively simple in terms of its principle consists in detecting, in sections, smears or other preparations originating from biopsies, an immunohistological type analysis. In this case, it therefore typically involves tests carried out on a crude sample (section of tumors consisting of more or less homogeneous cells). The application of this technique consists in demonstrating a reaction between the reagents and the vimentin on the preparation. The demonstration of complexes (detected proteins-reagents) means that it is necessary to detect a signal generated by using radioactive tracers or fluorescent reagents, for example, or calorimetric methods. The noninvasive tumor cells containing phosphorylated vimentin will show a positive reaction with the phosphorylated-vimentin-specific reagent selected. Conversely, the invasive cells that do not contain detectable amounts of modified vimentin will not show a signal.

Tests based on cell sorting using fluorescence (method referred to as flow cytometry or FACS, Fluorescent Activated Cell Sorting) (Hulett et al., 1969; Parks and Herzenberg, 1984): a reagent labeled with a fluorescent group may be used in order to label whole cells derived and isolated from biopsies and to make it possible to sort and quantify these cells positive for the presence of modified vimentin. In the case of this method, non-lysed cells dissociated from the freshly collected biopsy tissue are brought into contact with the selective fluorescent reagent and the suspension is then analyzed using a cell sorter. The cell sorter detects, individually, the intensity of the fluorescent signal associated with each cell, isolates the cells in specific reservoirs and counts the number of cells thus selected. Instruments designed for FACS analysis are distributed, for example, by the company Becton Dickinson (Franklin Lakes, N.J., USA; http://www.bd.com). The use of appropriate and optimized technical parameters (cell dilution, optical parameters, etc.) makes it possible to envision the selective sorting and counting of cells containing modified or nonmodified vimentin and to ensure the feasibility of this method of use of the invention described here. It should be noted, however, that a flow cytometry approach applied to the analysis of protein extracts (i.e. on cells that have been lysed this time) could be carried out, for example, with the Bio-Plex technology from Bio-Rad (Hercules, Calif., USA; http://www.bio-rad.com).

The two methods described above do not necessarily require the preparation of a cell extract since they can be applied directly to cells. Other methods (based especially on antibody-type reagents) listed hereinafter require, for their part, the preparation of a protein extract obtained after lysis of the sample to be analyzed. These methods are based on the analysis of the interaction between the reagent and the protein to be detected, optionally including the adsorption of the latter onto a substrate (ELISA methods, RIA, FRET, microarrays, detectors using surface plasmon resonance, etc.). The generation of a signal associated with this interaction allows the detection of modified vimentin in a sample and its quantification, according to the intensity of the signals generated. Thus, antibodies directed against the modified vimentin can be used to selectively interact with (or even, in certain cases, capture) the protein of interest in a complex mixture consisting of an extract containing virtually all the proteins contained in a tumor biopsy. The interaction between the antibody and the modified vimentin results in the formation of a binary complex, referred to as immunocomplex, composed of modified vimentin and of antibodies. In most cases, and as for the detection method preferred by the authors, which is described in this document, the methods presented hereinafter refer more particularly, but not exclusively, to biopsy sample lysates.

Tests based on isotopic labeling and RIA (radioimmuno-assays) (Yalow and Berson, 1960; Booth et al., 1982): In one of the variants of this assay, the immunocomplex is produced by adding, to the reaction medium, in addition to the biopsy protein extract of the antibody, a known amount of modified vimentin labeled with a radioactive isotope. The modified vimentin present in the biological extract and the radioactive-tracer vimentin will compete for the binding of the antibody. After selection of the immunocomplexes formed, the amount of radioactivity detectable in the fraction thus isolated is inversely proportional to the amount of modified vimentin present in a biopsy sample. Diagnostic kits using the RIA principle are distributed, for example, for various assays, by the company Schering/Cis-Bio International (Gif/Yvette, France; www.cisbiointernational.fr).

Tests based on fluorescence transfer methods, FRET (Fluorescence Resonance Energy Transfer) : For such an analysis, the assay can, in its most appealing version, be carried out directly in solution (homogeneous-phase assay) and does not require the isolation or purification of one or other of the components of the immunocomplex. This method requires, in one of these variants, the use of two different antibodies directed against the modified vimentin and labeled with appropriate fluorescent groups. The two fluorescent groups are selected in such a way that their optical characteristics make it possible for one of the groups to be excited by the light radiation used for measuring fluorescence, and then allow the transfer of the excitation energy to the second fluorescent group, which emits, in the last instance, a fluorescence radiation of very specific wavelength. The fluorescence transfer is only effective if the two molecules are maintained in sufficiently close proximity to one another. In fact, in this type of assay, the two antibodies labeled with the two fluorescent groups are chosen so as to be able to bind simultaneously to the modified vimentin. The ternary immunocomplex formed (excitation fluorescence antibody-vimentin-light emission fluorescent antibody) therefore allows two antibodies to be brought closer and, in this case only, a fluorescence signal can be detected. The intensity of the fluorescence signal measured is therefore directly proportional to the amount of modified vimentin present in the biological extract (Mathis, 1995; Szollosi et al., 1998; Blomberg et al., 1999; Ueda et al., 1999; Enomoto et al., 2000). The analysis of proteins by the fluorescence transfer method can be carried out on the Kryptor® device from the German company B.R.A.H.M.S. (www.brahms.de).

Detection Tests Based on the Macroscopic Demonstration of Immunocomplexes (Latex Test, Immunodetection Bands)

(Singer et al., 1957; Hechemy et al., 1974): In this type of detection system, anti-modified vimentin antibodies are chemically coupled to particulate components of micrometric size such as colored or colorless polymer beads. Incubation, in a liquid medium, of a fluid suspension of these antibody-coated beads, with the biological extract to be analyzed, results in the creation of immunocomplexes that aggregate several vimentin molecules and several polymer beads. This aggregation is reflected by the formation of packets of beads, the size of which becomes macroscopically large to the point of being visible “to the eye” by an operator. In a common and complex variant of the system, the immunocomplexes are subjected to migration by capillarity on a chromatography support strip and reveal through the creation of a colored strip indicating the presence or the absence of the antigen detected (test symbolized by immunodetection strips largely used routinely for example for pregnancy tests). Latex tests are marketed by the company Bio-Merieux for microbiological diagnosis for example (www.biomerieux.com).

Tests involving capture on a substrate: Various tests for detecting antigen-antibody reactions can be carried out using various methods of interactions or adsorptions of the components with substrates, reaction cupules or microdetection systems. These tests very commonly ally high levels of performance, associated with the high sensitivities of the methods, with relatively simple handling of the samples, soundness of the tests, and capacities for high-throughput processing of numerous samples simultaneously.

a) ELISA-derived methods. One of the now conventional methods, called ELISA (Enzyme Linked Immunoassays) (Engvall and Perlmann, 1971, 1972; Engvall et al., 1971) consist in bringing about the creation of immuno-complexes in a form immobilized on the walls of the wells (cupules) of plastic multiwell assay plates. This type of method comes in a multitude of variants depending on whether the protocols are based on first immobilization of antibodies or of antigens in the bottom of the wells and depending on the visualizing methods used (direct or indirect ELISA). By way of example, it may be briefly mentioned that this type of test can be carried out according to the description which follows for the detection of the modified vimentin. Thus, using the protein extract derived from the biopsy, the wells of the ELISA assay plate are filled with increasing dilutions of the sample. The various proteins of the extract bind by adsorption to the bottom of the wells. After washing of the wells, the proteins that adhere to the walls of the wells are brought into contact with the specific antibodies intended to detect the presence of the modified vimentin. As a result, the anti-modified vimentin antibody will be immobilized in the bottom of the well, if and only if the antigenic protein is adsorbed onto the wall of the cupule. A detection step (based, for example, on a simple calorimetric test) for the presence of antibody at the bottom of the wells consequently provides information on the presence of modified vimentin, adsorbed in the wells, and then on its presence in the starting sample. By carrying out assays on dilutions of the sample, it is possible to quantitatively estimate the amount of modified vimentin in the biological sample. The ELISA assay analyses can be carried out on devices such as the VIDAS system distributed by Bio-Mérieux (www.biomerieux.com).

b) Methods based on “protein microarrays”. In a similar immunocomplex immobilization approach, the microarray technique is based on a logic of miniaturization, automation and more or less massive parallelization of the number of tests. For these tests, it is necessary to create “protein microarrays” consisting of generally planar solid surfaces (glass slides, silicon fragments, etc.) comprising antibodies bound by various chemical processes to the substrate, each antibody being deposited onto a small surface area of the substrate representing a few square microns of surface area. For example, the antibodies are immobilized on the substrate by depositing microdrops of antibody suspension onto these substrates (Peluso et al., 2003, Kusnezow and Hoheisel, 2003). After incubation with the samples to be analyzed, the immuno-complexes formed can be detected by various technical means, the most common being based on the detection of fluorescence signals, or by a method using surface plasmon resonance (Vikinge et al., 1998; Kusnezow and Hoheisel, 2003).

Surface plasmon resonance is based on a well known experimental physical principle. The planar surface of a film of gold reflects an incident light ray in a direction predicted by the laws of conventional optics. Nevertheless, for a very small part of the light beam reflected, under a certain incidence, a significant decrease in the number of photons reflected is observed. The angle of incidence of the reflection zone of the less luminous zone depends on the amount of material attached to the face of the gold film opposite the face irradiated by the incident light beam. Any antibody or antigen interaction or constitution of immunocomplexes on the opposite face of a detector based on plasmon resonance and irradiated under a certain incidence by a light ray therefore causes a noticeable change in the angle of reflection of the less luminous part of the reflected light beam. The detection of this deflection of the angle of reflection makes it possible to demonstrate and quantify the attachment of components on the detector.

The latter method of detection forms the basis of a technique for analyzing interactions between antibodies and proteins and makes it possible to measure the kinetic parameters for interaction between antigens and antibodies, just as it does the amounts of antigens present in a sample (Fagerstam et al., 1990; Szabo et al., 1995). This technique is developed in the form of automated measuring devices, an example of which is known under the name BIAcore (BIAcore AB, Uppsala, Sweden; http://www.biacore.com). It is interesting to mention, by way of example, that the use of this type of detector for detecting phenomena of association of vimentin peptide fragments with one another, and also the demonstration of the impact of phosphorylation on these interactions, was the subject of a recent scientific publication (Gohara et al., 2001).

c) Method based on mass spectrometry: Finally, and quite naturally, detection of the modified vimentin by virtue of the SELDI technique can be provided by using antibodies immobilized on affinity substrates that can be used on the Ciphergen mass spectrometer or any other mass spectrometer suitable for this type of analysis. It is in fact possible to immobilize antibodies by chemical grafting onto silica substrates or polymer substrates and to use these substrates to trap the modified vimentin which is present in a sample to be analyzed. The immunocomplexes thus immobilized on the substrates can subsequently be analyzed by mass spectrometry. In this type of analysis, the peak of the modified and/or nonmodified vimentin protein (all depends on the antibodies grafted onto the substrate) can be detected in the mass spectrum. The use of antibodies grafted onto the substrate makes it possible to constitute an extremely sensitive and specific assaying method. Given the affinity of the antibodies for the protein, minute amounts of vimentin present in a sample may be detected; in addition, the perfect selectivity of the antibodies with respect to the protein against which they are directed guarantees the complete specificity of the assay.

d) Method based on Western blotting (Towbin et al., 1979; Burnette 1981). In the case of this method, the protein extract is analyzed by polyacrylamide gel electrophoretic migration in a denaturing medium (technique known, in one of its variants, under the acronym SPAGE: for SDS polyacrylamide gel electro-phoresis; or polyacrylamide gel electrophoresis in the presence of the denaturing detergent Sodium Dodecyl Sulfate). This electrophoretic migration makes it possible to separate proteins as a function of their respective molecular masses (Laemmli 1970). At the end of this separation, very conventional “Western blotting” protocols make it possible to immunodetect, within the electrophoretic profile, the protein of interest using an antibody directed against this protein. These protocols make it possible to reveal the presence of the protein of interest by virtue of radio-active, fluorescent or luminescent techniques. The analysis can be carried out in such a way as to obtain a quantitative estimation of the amount of protein in the starting sample that is sufficiently accurate to provide a diagnosis of clinical interest (Procaccio et al., 1999). The implementation of this test using an anti-modified vimentin antibody will result in the visualization, on the Western blotting profile and for the samples which contain modified vimentin, of a signal present in a zone of molecular mass corresponding to the modified vimentin. Tests of this type could be carried out using the equipment required for Western blotting distributed by Immunetics (Boston, Mass., USA; http://www.immunetics.com).

If need be, if the electrophoretic migration conditions can be optimized such that, given the different physicochemical characteristics (different pI and molecular masses) of vimentin and of phosphorylated vimentin, these two components can be separated on two different zones of the gel, then the detection could, in this case, require just one antibody. In fact, in this case, an antibody that recognizes both the phosphorylated form and the nonphosphorylated form will result in one or two vimentin detection signals showing up on the Western blot, each of the signals then being readily attributable to the natural vimentin or the phosphorylated vimentin, depending on the migration distances of these two entities observed during the electrophoresis in the polyacrylamide gel.

In all the methods cited above, mention is made of the use of antibodies. This preferred description of the use of antibodies in the methods described does not exclude the fact that the tests mentioned here can also be carried out with other reagents capable of interacting with modified or nonmodified vimentin, such as, by way of example, aptamers.

Example of an electrophoretic migration test with demonstration of posttranslational modifications of vimentin, not using antibodies for the detection: in a manner similar to the “Western blotting” method described above, it is possible to separate the proteins in a polyacrylamide gel and then, after migration, to stain this gel and its footprint using specific chemical reagents. In this case, the use of a reagent which selectively binds to phosphate groups present on proteins or induces specific chemical reactions with these phosphate groups will make it possible to produce, depending on the case, a radio-active, luminescent, calorimetric (in the case of the coloring reagent included in the “GelCode® phospho-protein staining kit”—Pierce, Rockford, Ill., USA; http://www.piercenet.com) or fluorescent (in the case of the fluorochrome included in the “Pro-Q® Diamond phosphoprotein gel stain” kit—Molecular Probes, Eugene, Oreg., USA; http://www.probes.com) signal. The creation of this signal will provide specific information regarding the presence of phosphate groups in the proteins separated in the electrophoresis gel. The detection of this signal revealing the presence of phosphate groups in a protein which shows a migration distance in the gel compatible with the molecular mass expected for vimentin will be sufficient information to allow a diagnosis of the presence of modified vimentin in the biological sample.

As described above, the selective detection of the modified vimentin can therefore also be obtained by interaction with antibodies or other reagents capable of interacting specifically with vimentin or by demonstrating original and distinguishable physico-chemical characteristics of the protein.

Moreover, as a supplement, and as is already the case with regard to the Western blotting method, it is possible to envision carrying out tests that make it possible to reveal or obtain the evaluation of two or more parameters which, combined, make it possible to confirm the presence of phosphorylated vimentin in a sample. These parameter combinations may, for example, be: size and the detection of phosphate groups; size and detection using components that interact specifically with vimentin; isoelectric point and size of the protein, etc. Two-dimensional electrophoresis analyses can potentially be carried out by using the instruments and reagents of the Multiphor™ range for example, developed by Amersham (http://www1.amershambiosciences.com).

In addition, considering that phosphorylated vimentin constitutes a very informative marker that makes it possible to give a prognosis of the favorable or unfavorable progression of a tumor, the method of diagnosis and prognosis of a pathology can include or be based on the detection or even the assaying of the enzymatic activities of various catalysts of cell metabolism, such as, in particular, the kinases and/or phosphatases that are involved in the process of posttranslational modification of vimentin by phosphorylation.

It is in fact entirely possible to envision imagining a method of diagnostic and prognostic interest that is not necessarily based on detecting only phosphorylated vimentin, but on the essential or even specific components that contribute to the modification and/or the stability of vimentin in its phosphorylated form. In this case, the diagnostic method would be based on the detection of one or more of the cellular components which acts in this vimentin modification process (these components are to be sought in a nonexhaustive list of the following factors: kinases; phosphatases; proteases; transporters that facilitate the presentation of vimentin to kinases in specific cellular compartments; transporters that ensure the sequestration and, consequently, the stability of phosphorylated vimentin in specific subcellular or even extracellular compartments).

By way of example, the assay, in the biopsy samples or other biological fluids, of the phosphorylation activity of the kinase(s) which phosphorylate(s) vimentin could show a zero or normal activity of these kinases in the case of invasive tumors and, on the contrary, a more or less strong increase in the detectable phosphorylation activity in the case of noninvasive tumors. Conversely, and according to the same logic as above, it is easy to predict that the assay of phosphatases which eliminate the phosphate groups that modify the amino acids of vimentin could show normal activities in the case of noninvasive tumors and, on the contrary, an increase in the detectable dephosphorylation activity in the case of invasive tumors.

In addition, not only could the measurement of the enzymatic activities of these compounds constitute a diagnostic method for tumor invasiveness, but the physical demonstration of these compounds, coupled to the quantitative estimation thereof, would just as much constitute an acceptable method of analysis provided that: either the amount of these components is modified in the cell, or one or other of their physicochemical characteristics is altered in these invasive or noninvasive tumor cells and these alterations can be detectable using an appropriate technique.

Here also, mention may be made of a detection method based on the revelation of the phosphorylation capacities specific to a tumor sample:

i.e.: a certain number of peptides known to be kinase targets can be deposited onto a microarray type substrate. Various target peptides AND the peptides corresponding to the vimentin peptides where the phosphorylations occur (detected in the context of the present invention) can be deposited onto this microarray. By incubating this microarray with a cell lysate to be analyzed, in the presence of radiolabeled ATP, the ability of the, biological sample to phosphorylate some peptides or other will be revealed. As a result, the qualitative (and semiquantitative) composition of the sample is identified, overall, in terms of these active kinase type components.

A “phosphorylome” of the sample is thus produced. The existence of vimentin-specific kinases (or in altered amount compared with a normal tissue reference) will make it possible to establish a prognosis of invasiveness or noninvasiveness of the tumor of which a sample was studied with this method.

The method according to the invention can be used for determining the aggressiveness and/or invasiveness of any type of tumor associated with an overexpression of vimentin, in particular central nervous system tumors, hormone-independent breast carcinomas (epithelial tumors), cervical carcinomas, kidney carcinomas, prostate cancers, bone metastases, hepatocarcinomas, melanomas or breast cancers.

Advantageously, the method according to the invention is used for determining the aggressiveness and/or invasiveness of central nervous system tumors, in particular astrocytomas and meningiomas.

The present invention also relates to a diagnostic kit for carrying out the method according to the invention, characterized in that it comprises, on an appropriate substrate, a means for detecting nonphosphorylated vimentin and/or a means for detecting phosphorylated vimentin.

According to a first embodiment of the invention, the means for detecting nonphosphorylated vimentin is chosen from:

For the tests based on the use of mass spectrometry, the kit will require affinity substrates for capturing the protein (example: ProteinChip® arrays from Ciphergen). These substrates will be adjusted according to the method of capture chosen: substrates with chemical reactivity suitable for the attachment of vimentin, or monoclonal or polyclonal antibodies that recognize all or part of the protein or of peptides thereof. Reagents of phosphatase and/or protease type will also be necessary depending on the implementation protocol chosen.

For the other tests, in particular ELISA assays, polyclonal or monoclonal antibodies that react against all or part of vimentin will be essential. Specific reagents for detecting the immunocomplexes formed during the test will be chosen from conventional detection systems suitable for tests of this type (for example: secondary antibodies coupled to enzymatic systems for implementing a calorimetric reaction). Reagents of phosphatase and/or protease type will also be necessary depending on the implementation protocol chosen.

For the radioimmunoassays, in addition to the antibodies (or other capture agents specific for vimentin and derivatives thereof), a tracer that is identical to vimentin, or that mimics the latter with respect to the capture agents, will be required in radioactively labeled form (or, optionally, labeled according to other methods, for example: fluorescently labeled).

According to another embodiment of the invention, the means for detecting phosphorylated vimentin is chosen from:

For the tests based on the use of mass spectrometry, the kit will require affinity substrates for capturing the protein (for example: ProteinChip arrays from Ciphergen). These substrates will be adjusted according to the method of capture chosen: substrates with chemical reactivity suitable for the binding of vimentin, or monoclonal or polyclonal antibodies that recognize all or part of the protein or of peptides thereof. Reagents of protease type will also be necessary according to the implementation protocol chosen.

For the other tests, in particular ELISA assays, polyclonal or monoclonal antibodies that react against all or part of vimentin will be essential. Specific reagents for detecting the immunocomplexes formed during the test will be chosen from conventional detection systems suitable for tests of this type (for example, secondary antibodies coupled to enzymatic systems that allow a calorimetric reaction to be carried out). The protease-type reagents will also be necessary according to the implementation protocol chosen. For Western blotting: antibodies specific for the protein or for peptides thereof, or calorimetric or fluorescent labeling reagents specific for phosphoryl groups.

For the radioimmunoassays, in addition to the antibodies (or other capture agents specific for vimentin and derivatives thereof), a tracer that is identical to vimentin, or that mimics the latter with respect to the capture agents, will be required in radioactively labeled form (or, optionally, labeled according to other methods, for example: fluorescently labeled).

Advantageously, the substrate is chosen from substrates of chemical compounds capable of interacting with phosphorylated or nonphosphorylated vimentin (for example: silica substrates with an ion exchange capacity), plastic multiwell assay plates, polyacrylamide gels or other matrix for separating proteins according to their physicochemical characteristics (molecular mass, ionic charges, etc.), latex beads or other materials suitably activated by reagents that allow interactions with the vimentin or the immunocomplexes.

In a manner obvious to those skilled in the art, the diagnostic kit may also include chemical reagents of any type that are essential to the extraction, solubilization, enrichment and enzymatic digestion of the phosphorylated or nonphosphorylated protein and also for the demonstration of the protein (staining, chemical modifications, etc.). The kit will also contain, for quantification and positive-control requirements, suitable standard proteins that may or may not be related to phosphorylated or nonphosphorylated vimentin.

The present invention also relates to a method for identifying an anticancer agent that makes it possible to reduce the aggressiveness and/or invasiveness of a tumor by promoting vimentin phosphorylation, characterized in that it comprises the steps consisting in:
culturing cells that produce nonphosphorylated vimentin,
bringing said cells into contact with an agent to be tested,
analyzing the vimentin produced in order to determine whether it is phosphorylated or nonphosphorylated after culturing of the cells in contact with the agent to be tested,
selecting the agent(s) capable of inducing vimentin phosphorylation in the cancer cells tested.

Those skilled in the art will be able to determine the means necessary for implementing the method according to the tested agents capable of acting on vimentin phosphorylation: “small molecules”, siRNAs, antisenses, etc.

Advantageously, the cells that produce vimentin are cancer cells. They may also be noncancer cells that express vimentin or else recombinant cells, more particularly mammalian cells, comprising a vector for the expression of vimentin or fragments thereof.

Also advantageously, the analysis of the vimentin produced, that makes it possible to determine whether it is phosphorylated or nonphosphorylated, is carried out by means of the method defined above.

Finally, the present invention relates to a method for limiting the aggressiveness and/or invasiveness of tumors, in which method vimentin phosphorylation is promoted in cancer cells by administering a suitable therapeutic agent.

It relates to the use of a suitable agent for promoting vimentin phosphorylation in cancer cells, for the preparation of a medicament for use in the treatment of cancerous tumors.

Various means can be used for this purpose, according to various therapeutic axes:
1. The first strategy that can be envisioned consists in intervening directly on the cellular concentrations of the various forms of vimentin. In this perspective, 3 axes of intervention are possible:
   a. directly decreasing the amount of natural (nonphosphorylated) vimentin synthesized by these cells or reducing the bioavailability thereof within the cell and thus making it possible to increase the overall yield of enzymatic phosphorylation of the cellular vimentin. This can be achieved by modifying the level of vimentin expression or by sequestering, or even “neutralizing”, the nonphosphorylated vimentin. The latter possibility would be based on the use of compounds which will complex and “mask” this protein, or on the control of the targeting of the protein outside the cytosolic compartment (routing into specific subcellular compartments or, optionally, secretion). The objective of these two independent processes is to remove the natural vimentin from any involvement in the biological phenomena of the cell;
   b. promoting vimentin accessibility to kinases. By acting on the availability of certain cellular components that interact naturally with vimentin, it may be possible to stimulate the phosphorylation of this protein;
   c. increasing the stability and the lifetime of phosphorylated vimentin by impeding the cellular systems that participate in the degradation or in the cytoplasmic elimination thereof.
2. A second possibility consists in directly promoting the action of the kinases that phosphorylate vimentin. This can be envisioned in two different ways: by increasing the specific enzymatic activity of these kinases, or through the overproduction thereof.
3. The third approach, which is capable of promoting the accumulation of phosphovimentin, is based, unlike the strategy described in point number 2 above, on various actions intended to reduce the level of cellular phosphatases or the activity thereof.

In the interest of optimal therapeutic effectiveness, a combination of two or more of these approaches is not of course excluded.

Strategy 1: Modification of Parameters Relating to Vimentin (Level of Expression, Accessibility, Stability)

In the context of this strategy, it is technically possible to envision reducing the cellular concentration of vimentin by more or less partially blocking the expression of the gene by various means. One possibility is based on negative regulation of the expression of the gene; the other is based on activating the degradation of the messenger RNAs (mRNAs) and on reducing the efficiency of the translation thereof.

Various pharmacological compounds or molecules can prove to be capable of more or less selectively reducing the efficiency of transcription of the vimentin gene. This may be obtained in particular by quantitatively or qualitatively modifying the role played by essential genetic regions on the rate of transcription of the vimentin gene (promoter regions, enhancers, silencers, etc.). By way of example, compounds such as retinoic acid derivatives have already been shown to be capable of impeding the transcription of the vimentin gene, and retinoic acid derivatives (N-(4-hydroxyphenyl) retinamide or 4-HPR) appear to block prostatic carcinogenesis (Webber et al., 1999).

The control of the expression of the gene can also be imagined by impairing the stability and/or the possibilities of translation of the mRNA, a product of normal transcription of the gene.

This strategy involves, for example, the use of one or more antisense polynucleotides capable of interacting, in the cell, with the mRNA and of disturbing its stability or its ability to be translated into vimentin by ribosomes. The use of antisense polynucleotides has already been experimented with in vitro in order to reduce vimentin expression. Thus, an overexpression of the vimentin gene has been observed in a hormone-insensitive and invasive prostate tumor cell line. Transfection with an antisense vimentin construct effectively reduces the invasive nature of these tumors, measured in vitro (Singh et al., 2003). This type of antisense vimentin oligonucleotide construct also reduces the rate of migration of epithelial cells during wound healing (Gilles et al., 1999). Similarly, the in vitro transfection of malignant mammary carcinoma cells with antisense oligonucleotides intended to block vimentin expression contributes to reducing the invasive activity of the cells thus treated (Hendrix et al., 1997). In the studies mentioned above, the impact of the antisense constructs or polynucleotide compounds on the amount of vimentin phosphorylation was not determined.

The appropriate use of siRNAs (small interfering RNAs) (Fire et al., 1998; Bernstein et al., 2001; Elbashir et al., 2001a and b), judiciously selected according to the primary sequence of the vimentin-encoding transcript, is of course a solution in terms of expected effectiveness for decreasing the amount of transcript available in the cell for vimentin synthesis.

Finally, according to a third option, the stability of the mRNA can also be reduced by intervening with respect to the concentration and/or the affinity of the cellular compounds that interact with the vimentin mRNA. In fact, the 3′ noncoding region of the vimentin transcript interacts with proteins such as HAX-1 and eEF1-gamma (Al-Maghrebi et al., 2002). It is acknowledged that the binding of proteins to noncoding regions of mRNAs is able to substantially modify the stability of the mRNAs (Saini et al., 1990; Cuadrado et al., 2002). It is therefore entirely plausible to envision that modifications to the concentrations of these proteins or the use of chemical molecules having the property of modifying the force of interaction of these proteins with the vimentin mRNA may indirectly affect the stability of the transcript and, consequently, result in a decrease in vimentin synthesis.

Another possibility consists in creating, in the cells, complexes between vimentin and varied molecular compounds so as to limit the amount of free non-posttranslationally-modified vimentin and, as a result, facilitate a considerable rate of phosphorylation of this protein by kinases, this being a final event that is expected, at the cellular level, to result in a limitation of tumor invasiveness. The molecules that could interact with the nonmodified vimentin are chemical molecules, antibodies or aptamers (polypeptide or polynucleotide molecules capable of binding with high affinity to a protein, in this instance vimentin). Vimentin is an antigenic molecule and it is entirely possible to produce antibodies directed against this protein. Studies such as those published by Hartmann et al. (2004) show, moreover, that vimentin can even give rise to autoimmune reactions since autoantibodies directed against vimentin can be detected in the serum of many patients suffering from skin lymphomas.

Intervention with respect to vimentin sequestration in subcellular compartments or the modification of its excretion by transmembrane pumping systems still remains a working hypothesis. However, this approach constitutes an interesting pathway since it has been reported that macrophages are found to be capable of secreting phosphorylated vimentin (Mor-Vaknin et al., 2003). Transporter systems capable of transporting vimentin from the cytosol out of the cells therefore clearly exist.

It is also possible to envision modifying the methods of interaction of normal cellular compounds with vimentin. In this case, it involves modifying the interactions between vimentin and the normal cellular proteins or compounds with which it naturally forms complexes.

By way of example, it may be mentioned that vimentin can interact with the tumor cell protein known as 14-3-3. The association of 14-3-3 with phosphovimentin appears to impair the dephosphorylation of the latter (Tzivion et al., 2000). As a result, any action capable of disturbing the interaction of vimentin with this 14-3-3 protein (or other compounds that bind to vimentin) will have the potentiality of facilitating phosphovimentin accumulation and therefore, consequently, of controllably reducing tumor invasiveness.

Finally, any of the cellular elements, such as proteases, that would specifically degrade phosphorylated vimentin, or membrane transporters that would specifically excrete vimentin, would represent targets against which to dedicate molecules or approaches intended to block such activities. The inhibition of these activities could, overall, tend to decrease the cellular concentration of phosphorylated vimentin.

The second strategy involving activation of vimentin phosphorylation activity is based on the development of molecules capable of interacting directly or indirectly with kinases and stimulating their specific activity. It is interesting to observe, in this perspective, that the treatment of CHO-K1 cells (cells of fibroblast origin made perpetual, from a Chinese hamster ovarian biopsy) with cAMP (cyclic adenosine 3′,5′-mono-phosphate) derivatives induces loss of the malignant phenotype. This event goes together with vimentin phosphorylation (Chan et al., 1989). It can be supposed that, in the case of this example, the cAMP could be held responsible, without this having been definitely demonstrated, for the activation of cAMP-dependent kinases. Moreover, the increase in the amount of cellular kinases can be increased by various approaches. For example, molecular biology technologies offer the possibility of creating gene constructs for the expression of kinases in tumor cells after these constructs have been introduced into said cells.

Finally, as regards the third strategy, phosphatases can constitute a molecular target of choice. Specifically, any slowing down of the activity of these enzymes should result in the accumulation of phospho-vimentin. Specific molecules, whose purpose is to inhibit phosphatases, will be all the more effective because phosphatases specifically involved in vimentin dephosphorylation will have been identified. The inhibition of phosphatases may be based on the use of chemical molecules capable of interacting with these enzymes or the use of iRNAs or antisense constructs capable of disturbing the expression of these enzymes. By way of example, it may be mentioned that it has been shown that calyculin A, which blocks phosphatase type 1, promotes the detection of a form of vimentin phosphorylated by the Rho kinase on serine 71 of vimentin (Inada et al., 1999). The possibilities of impeding the activity of phosphatases may also involve interventions with respect to the formation of active multimolecular phosphatase complexes. Any action on the compounds which activate phosphatases is liable to prevent the activation of these enzymes. It has been demonstrated, for example, that the B55 protein, which activates the PP2A phosphatase, is required for vimentin dephosphorylation (Turowski et al., 1999).

The implementing examples below make it possible to illustrate the invention more clearly, without, however, seeking to limit the scope thereof.

### EXAMPLE 1

### Detection of Phosphorylated Vimentin for the Diagnosis of Various Invasive or Noninvasive Solid Tumors According to the Embodiment Based on SELDI-Type Mass Spectrometry

The tissue samples, in the form of frozen biopsy sections, are used to prepare the protein extracts and are analyzed according to the protocols described hereinafter: tissue sections 20 μm thick are cut on a microtome and 20 sections are incubated for 30 minutes at 4° C. in 300 microliters of a lysis buffer (Reporter Lysis Buffer, Promega, Madison, Wis., USA) containing a conventional mixture of antiproteases. The sample is vortexed several times during this incubation period. After centrifugation (10 000 g for 10 min at 4° C.), the supernatant (referred to as final extract) is obtained and a protein assay carried out by a conventional method makes it possible to verify that the protein concentration in the final extract reaches a titer of 2 to 3 micrograms of total proteins per microliter.

The final protein extracts are analyzed by means of SAX2 ProteinChip® analytical arrays from the company Ciphergen, composed of active surfaces made up of an “anion exchange” matrix. In order to ensure equilibration of the ionic charges of the active surfaces, the SAX2 ProteinChips® are impregnated with a fixing buffer (100 mM Tris, pH 8.0, and 0.1% Triton X100) for 5 minutes. After removal of this buffer, the final protein extracts are deposited onto the active surfaces in the following way: the extracts are diluted to a final titer of 0.1 microgram per microliter and 100 microliters of this dilution are placed in contact with the active surfaces. The ProteinChips® are then incubated at ambient temperature for 45 minutes and subjected to continuous vigorous agitation. After incubation, the ProteinChips® are washed twice using a buffer consisting of 100 mM Tris, pH 8.0, and 0.1% Triton X100, and then once with a second washing buffer composed of 100 mM Tris, pH 8.0. A final wash is carried out in 2 mM HEPES buffer, pH 7.5. Finally, the active surfaces are dried under a stream of air and 1.6 microliters of an SPA solution (Sinapinic acid, Ciphergen) is deposited onto these surfaces.

The ProteinChips® are analyzed using the Ciphergen mass spectrometer. The device is subjected to a calibration step using known purified protein standards deposited onto ProteinChips®. The standards normally used are bovine insulin (molecular mass 5733.6 Da), bovine cyto-chrome c (12 230.9 Da) and bovine serum albumin (66 410.0 Da).

The analysis of biopsy samples according to the method described above was carried out on invasive and noninvasive tumors of meningioma type (35 tumors), on lung tumors (4 samples) and on central nervous system glial cell tumors (of the glioblastoma and oligodentro-glioma type, 4 samples). Phosphorylated vimentin is detected by the presence of a peak at 53.5 kDa.

The analytical results are given in table 1 (values calculated over a collection of 35 different tumors).

| | Intensity of the mass peak | |
|---|---|---|
| | detected at 53.5 kDa (mean | |
| Sample type | of the intensities in | |
| (number) | arbitrary units) | P-value |
| Extract of non- | 6.5 | 0.00024 |
| invasive tumor (17) | | |
| Extract of invasive | 1 | |
| tumor (18) | | |

### EXAMPLE 2

### Detection of Nonphosphorylated Vimentin

The phosphorylated vimentin can be purified from the protein extracts by means of a protocol which consists in subjecting the extract to chromatography on a Q hyperD® column under operating conditions that facilitate the binding of vimentin in its phosphorylated form to the chromatographic substrate, and then in specifically eluting this protein with solutions that have a well defined composition. The extract is incubated with the column resin in the presence of 100 mM Tris, pH 8.5, for 30 min with agitation. Three washes of 5 min in 100 mM sodium citrate buffer, pH 3.5, with agitation are carried out, followed by 3 washes in a solution composed of 5% acetonitrile +0.5% trifluoroacetic acid +50% isopropanol. The elution of the protein of interest is carried out in 16% acetonitrile +0.1% trifluoroacetic acid +33% isopropanol.

After purification of the phosphorylated protein, an incubation in the presence of a phosphatase, according to conventional methods (for 2 hours at ambient temperature in the presence of alkaline phosphatase extracted from calf intestine) makes it possible to eliminate the phosphate groups. The nonphosphorylated vimentin can be detected by SELDI mass spectrometry.

Phosphorylated vimentin is capable of interacting extremely strongly with an anion exchange matrix of the SAX2 ProteinChip® type. The adsorption of the vimentin onto the matrix withstands treatments with buffers of acidic pH and the bound protein is not eluted, in particular by washing at pH 3.5. After phosphatase treatment of the phosphorylated vimentin purified from the noninvasive tumors, the dephosphorylated vimentin loses its anionic nature and is no longer capable of adsorption to the SAX2 anion exchange matrix. It remains, however, capable of binding to a hydrophilic matrix (Ciphergen ProteinChip® NP20).

The results of analysis by mass spectrometry after binding to the Ciphergen ProteinChip® substrate of SAX2 and NP20 type are indicated in table 2.

| | ProteinChip ® substrate | |
|---|---|---|
| | selected for SELDI | |
| | analysis | |
| | SAX2 ProteinChip ® | |
| | (anion exchange | |
| | surface) | |
| | Detection of the | NP20 ProteinChip ® |
| | mass peak at | (hydrophilic |
| | 53.5 kDa of | surface) |
| | phosphorylated | Mass of the peak |
| Sample type | vimentin | detected (in kDa) |
| Purified | present | 53.5 |
| phosphorylated | | |
| vimentin | | |
| Purified phosphorylated | absent | 53.2 |
| vimentin having | | |
| undergone phosphatase | | |
| treatment | | |

### EXAMPLE 3

### Detection of Enzymatic Digestion Fragments of Vimentin

The vimentin can be detected using proteolytic fragmentation of the sample or of the purified phosphorylated vimentin (see example 2). In this case, the identification of the protein is based on a strategy consisting in analyzing, by mass spectrometry, the result of the digestion of phosphorylated vimentin (or even vimentin dephosphorylated by prior treatment with phosphatases) with a protease (trypsin or Lys endoproteinase, or Glu-C endoproteinase, etc.) and in pinpointing the peptide peaks detected that comprise masses identical to those of the peptides predicted from the structure of vimentin.

Analysis of the residue from Glu-C endoproteinase digestion of phosphorylated vimentin shows, for example, 25 peptides of different molecular masses in a molecular-mass window of detection of between 1000 and 3000 Da.

The analysis of correspondence between the mass of these 25 detected peptides with those of the peptides that can be generated from vimentin showed that more than about twelve of these peptides corresponds strictly to peptides derived from the digestion of vimentin by Glu-C endoproteinase.

The analytical results are indicated in table 3.

TABLE 3

| | | | | | |
|---|---|---|---|---|---|
| Measured | Avg/ | Computed | Error | Residues | Narrow |

| | | | | | | | |
|---|---|---|---|---|---|---|---|
| Mass (M) | Mono | Mass | (Da) | Start | To | Cut | Peptide sequence |

| | | | | | | | |
|---|---|---|---|---|---|---|---|
| 1107.092 | A | 1106.330 | 0.762 | 397 | 405 | 0 | IATYRKLLE |
| 1356.192 | A | 1356.585 | −0.393 | 231 | 241 | 2 | IAFLKKLHFEE |
| 1356.192 | A | 1356.585 | −0.393 | 230 | 240 | 2 | FIAFLKKLHFE |
| 593.792 | A | 1593.670 | 0.122 | 355 | 368 | 0 | AANYQDTIGRLQDE |
| 1699.692 | A | 1698.816 | 0.876 | 301 | 315 | 0 | AANRNNDALRQAKQE |
| 1726.592 | A | 1726.990 | −0.398 | 231 | 244 | 3 | IAFLKKLHEEKIQE |
| 1943.292 | A | 1943.234 | 0.058 | 409 | 425 | 0 | SRISLPLPNFSSLNLRE |
| 2013.392 | A | 2013.284 | 0.103 | 110 | 125 | 0 | LNDRFANYIDKVRFLE |
| 2192.592 | A | 2192.411 | 0.181 | 135 | 153 | 3 | LEQLKGQGKSRLGDLYEEE |
| 2258.392 | A | 2258.516 | −0.125 | 406 | 425 | 2 | GEESRISLPLPNFSSLNLRE |
| 2258.392 | A | 2258.541 | −0.149 | 175 | 192 | 3 | RDNLARDIMRLRKKQEE |
| 2405.992 | A | 2405.607 | 0.385 | 201 | 221 | 0 | NTLQSFRQDVDNASLARLDLR |
| 2606.192 | A | 2605.801 | 0.391 | 199 | 221 | 1 | AENTLQSFRQDVDNASLARLDLE |
| 2917.992 | A | 2918.218 | −0.225 | 201 | 225 | 1 | NTLQSERQDVDNASLARLDLERKKE |

“Measured Mass,”: mass of the peptides detected
“Computed Mass”: masses of the peptides from endo Glu-C digestion predicted from the known sequence of vimentin
“Residues Start”: position of the first amino acid of the peptide detected within the vimentin sequence
“Residues To”: position of the last amino acid of the peptide detected within the vimentin sequence

### BIBLIOGRAPHICAL REFERENCES

Al-Maghrebi M, Brule H. Padkina M, Allen C, Holmes W M, Zehner Z E., Nucleic Acids Res. 2002, 30, 5017-28.
Ando S. Tanabe K, Gonda Y. Sato C, Inagaki M., Biochemistry. 1989, 28, 2974-9.
Ando S. Tokui T. Yamauchi T. Sugiura H. Tanabe K, Inagaki M., Biochem Biophys Res Commun. 1991, 175, 955-62.
Bernstein E, Denli A M, Hannon G J., RNA. 2001, 7, 1509-21.
Blomberg K, Hurskainen P. Hemmila I., Clin Chem. 1999, 45, 855-61.
Booth J C, Hannington G. Bakir T M, Stern H. Kangro H. Griffiths P D, Heath R B., J Clin Pathol. 1982, 35, 1345-8.
Burnette W N., Anal Biochem. 1981, 112, 195-203.
Cairncross J G, Ueki K, Zlatescu M C, Lisle D K, Finkelstein D M, Hammond R R, Silver J S, Stark P C, Macdonald D R, Ino Y, Ramsay D A, Louis D N., J Natl Cancer Inst. 1998, 90, 1473-9.
Cattoretti G, Andreola S, Clemente C, D'Amato L, Rilke F., Br J Cancer. 1988, 57, 353-7.
Chan D, Goate A, Puck T T., Proc Natl Acad Sci USA. 1989, 86, 2747-51.
Chatel M. and Brucher J.-M., in “Neuro-oncologie” [“Neurooncology“], Publisher J. Hildebrand, Doin 2001, p. 25-34
Chou Y H, Ngai K L, Goldman R., J Biol Chem. 1991, 266, 7325-8.
Chu Y W, Seftor E A, Romer L H, Hendrix M J., Am J Pathol. 1996, 148, 63-9.
Cuadrado A, Navarro-Yubero C, Furneaux H, Kinter J, Sonderegger P, Munoz A., Nucleic Acids Res. 2002, 30, 2202-11.
Daumas-Duport C. and Figarella-Branger D., in “Classification histopronostiques des tumeurs cérébrales gliales et leurs impacts en thérapeutique” [“Histoprognostic classification of glial brain tumors and impact thereof in therapy”, Publisher espaces 34, 2002; p 11-48)
Dierick A M, Praet M, Roels H, Verbeeck P, Robyns C, Oosterlinck W., Histopathology. 1991, 18, 315-22.
Donhuijsen K, Schulz S., Pathol Res Pract. 1989, 184, 287-91.
Elbashir S M, Lendeckel W, Tuschl T., Genes Dev. 2001a, 15, 188-200.
Elbashir S M, Harborth J, Lendeckel W, Yalcin A, Weber K, Tuschl T., Nature. 2001b, 411, 494-8.
Ellington A D, Szostak J W, Nature 1990, 346, 818-22
Engvall, P. Perlmann, Immunochemistry 1971, 8, 871-874
Engvall E, Jonsson K, Perlmann P., Biochim Biophys Acta. 1971, 251, 427-34.
Engvall E, Perlmann P., J Immunol. 1972, 109, 129-35.
Enomoto K, Aono Y, Mitsugi T, Takahashi K, Suzuki R, Preaudat M, Mathis G, Kominami G, Takemoto H., J Biomol Screen. 2000, 5, 263-8.
Fagerstam L G, Frostell A, Karlsson R, Kullman M, Larsson A, Malmqvist M, Butt H., J Mol Recognit. 1990, 3, 208-14.
Ferrari S, Battini R, Kaczmarek L, Rittling S, Calabretta B, de Riel J K, Philiponis V, Wei J F, Baserga R., Mol Cell Biol. 1986, 6, 3614-20.
Ferrari S, Cannizzaro L A, Battini R, Huebner K, Baserga R., Am J Hum Genet. 1987, 41, 616-26.
Fire A, Xu S, Montgomery M K, Kostas S A, Driver S E, Mello C C., Nature. 1998, 391, 806-11.
Gao C, Mao S, Lo C H, Wirsching P, Lerner R A, Janda K D., Proc Natl. Acad Sci USA. 1999, 96, 6025-30.
Gilles C, Polette M, Piette J, Delvigne A C, Thompson E W, Foidart J M, Birembaut P., J Pathol. 1996, 180, 175-80.
Gilles C, Polette M, Zahm J M, Tournier J M, Volders L, Foidart J M, Birembaut P., J Cell Sci. 1999, 112, 4615-25.
Gohara R, Tang D, Inada H, Inagaki M, Takasaki Y, Ando S., FEBS Lett. 2001, 489, 182-6.
Hartmann T B, Thiel D, Dummer R, Schadendorf D, Eichmuller S., Br J Dermatol. 2004, 150, 252-8.
Heatley M, Maxwell P, Whiteside C, Toner P., J Clin Pathol. 1995, 48, 1031-4.
Heatley M K, Ewings P, Odling Smee W, Maxwell P, Toner P G., Pathology. 2002, 34, 230-2.
Hechemy K, Stevens R W, Gaafar H A., Appl Microbiol. 1974, 28, 306-11.
Hendrix M J, Seftor E A, Seftor R E, Trevor K T., Am J Pathol. 1997, 150, 483-95.
Holck S, Pedersen L, Schiodt T, Zedeler K, Mouridsen H., Virchows Arch A Pathol Anat Histopathol. 1993, 422, 475-9.
Honore B, Madsen P, Basse B, Andersen A, Walbum E, Celis J E, Leffers H., Nucleic Acids Res. 1990, 18, 6692.
Huang T J, Lee T T, Lee W C, Lai Y K, Yu J S, Yang S D., J Protein Chem. 1994, 13, 517-25.
Hulett H R, Bonner W A, Barrett J, Herzenberg L A., Science. 1969, 166, 747-9.
Inada H, Togashi H, Nakamura Y, Kaibuchi K, Nagata K, Inagaki M., J Biol Chem. 1999, 274, 34932-9.
Kiernan J A, Histological and histochemical methods. Theory and practise. Third edition. Oxford: Butterworth-Heinemann 1999.
Knappik A, Ge L, Honegger A, Pack P, Fischer M, Wellnhofer G, Hoess A, Wolle J, Pluckthun A, Virnekas B., J Mol Biol 2000, 296, 57-86
Knight Z A, Schilling B, Row R H, Kenski D M, Gibson B W, Shokat K M., Nat Biotechnol. 2003, 21, 1047-54.
Kusnezow W, Hoheisel J D., J Mol Recognit. 2003, 16, 165-76.
Laemmli U K., Nature. 1970, 227, 680-5.
Lang S H, Hyde C, Reid I N, Hitchcock I S, Hart C A, Bryden A A, Villette J M, Stower M J, Maitland N J., Prostate. 2002, 52, 253-63.
Lilienbaum A, Legagneux V, Portier M M, Dellagi K, Paulin D., EMBO J. 1986, 5, 2809-14.
Mathis G., Clin Chem. 1995, 41, 1391-7.
Merchant M, Weinberger S R., Electrophoresis. 2000, 21, 1164-77.
Mor-Vaknin N, Punturieri A, Sitwala K, Markovitz D M., Nat Cell Biol. 2003, 5, 59-63.
Parks D R, Herzenberg L A., Methods Enzymol. 1984, 108, 197-241.
Peluso P, Wilson D S, Do D, Tran H, Venkatasubbaiah M, Quincy D, Heidecker B, Poindexter K, Tolani N, Phelan M, Witte K, Jung L S, Wagner P, Nock S., Anal Biochem. 2003, 312,. 113-24.
Perreau J, Lilienbaum A, Vasseur M, Paulin D., Gene. 1988, 62, 7-16.
Procaccio V, Mousson B, Beugnot R, Duborjal H, Feillet F, Putet G, Pignot-Paintrand I, Lombes A, De Coo R, Smeets H, Lunardi J, Issartel J P., J Clin Invest. 1999, 104, 83-92.
Raymond W A, Leong A S., J Pathol. 1989, 158, 107-14.
Rusnak F, Zhou J, Hathaway G M., J. Biomol. Techniques 2002, 13, 228-37.
Saini K S, Summerhayes I C, Thomas P., Mol Cell Biochem. 1990, 96, 15-23.
Seshadri R, Raymond W A, Leong A S, Horsfall D J, McCaul K., Int J Cancer. 1996, 67, 353-6.
Singer J M, Plotz C M, Pader E, Elster S K., Am J Clin Pathol. 1957, 28, 611-7.
Singh S, Sadacharan S, Su S, Belldegrun A, Persad S, Singh G., Cancer Res. 2003, 63, 2306-11.
Sommers C L, Walker-Jones D, Heckford S E, Worland P, Valverius E, Clark R, McCormick F, Stampfer M, Abularach S, Gelmann E P., Cancer Res. 1989, 49, 4258-63.
Szabo A, Stolz L, Granzow R., Curr Opin Struct Biol. 1995, 5, 699-705.
Szollosi J, Damjanovich S, Matyus L., Cytometry. 1998, 34, 159-79.
Towbin H, Staehelin T, Gordon J., Proc Natl Acad Sci USA. 1979, 76, 4350-4.
Tsujimura K, Ogawara M, Takeuchi Y, Imajoh-Ohmi S, Ha M H, Inagaki M., J Biol Chem. 1994, 269, 31097-106.
Tuerk C, Gold L, Science 1990, 249, 505-10
Turowski P, Myles T, Hemmings B A, Fernandez A, Lamb N J., Mol Biol Cell. 1999, 10, 1997-2015.
Tzivion G, Luo Z J, Avruch J., J Biol Chem. 2000, 275, 29772-8.
Ueda H, Kubota K, Wang Y, Tsumoto K, Mahoney W, Kumagai I, Nagamune T., Biotechniques. 1999, 27, 738-42.
Vikinge T P, Askendal A, Liedberg B, Lindahl T, Tengvall P., Biosens Bioelectron. 1998, 13, 1257-62.
Webber M M, Bello-DeOcampo D, Quader S, Deocampo N D, Metcalfe W S, Sharp R M., Clin Exp Metastasis. 1999, 17, 255-63.
Weinberger S R, Morris T S, Pawlak M., Pharmaco-genomics 2000, 1, 395-416
Yalow R S, Berson S A., J Clin Invest. 1960, 39, 1157-75.
Yang H Y, Lieska N, Shao D, Kriho V, Pappas G D., Mol Chem Neuropathol. 1994, 21, 155-76.
Yoon W H, Song I S, Lee B H, Jung Y J, Kim T D, Li G, Lee T G, Park H D, Lim K, Hwang B D., Cancer Lett. 2004, 203, 99-105.
Yung W K, Luna M, Borit A., J Neurooncol. 1985, 3, 35-8.

1. A method that can be used for determining the aggressiveness or invasiveness of a tumor, comprising:
preparing a biological extract comprising vimentin; and
determining whether the vimentin thus extracted is phosphorylated or nonphosphorylated.
wherein the sample of tumor cells is taken beforehand.

2. The method as claimed in claim 1, characterized in that it is determined whether the vimentin is phosphorylated or nonphosphorylated, by a method selected from the group consisting of:
SELDI-TOF analysis,
the FACS method or equivalents thereof,
RIA isotopic labeling,
FRET fluorescence transfers,
the demonstration of immunocomplexes,
methods for detecting antigen-antibody reactions, and
electrophoretic migration tests with demonstration of posttranslational modifications of vimentin.

3. The method as claimed in claim 1, characterized in that the tumor is a central nervous system tumor.

4. A diagnostic kit for carrying out a method as claimed in claim 1, characterized in that it comprises, on an appropriate substrate, a means for detecting nonphosphorylated vimentin or a means for detecting phosphorylated vimentin.

5. The kit as claimed in claim 4, characterized in that the means for detecting nonphosphorylated vimentin is selected from the group consisting of:
antibodies that react with all or part of the protein, reagents capable of interacting specifically with vimentin, reagents for modification or cleavage of the protein at the phosphorylation sites, and radio-active tracers.

6. The kit as claimed in claim 4, characterized in that the means for detecting phosphorylated vimentin is selected from the group consisting of:
antibodies that react with all or part of the protein, reagents capable of interacting specifically with vimentin, reagents for modification at the phosphorylation sites, reagents for specific cleavage of the protein at the phosphorylation sites, and radioactive tracers.

7. The kit as claimed in claim 4, characterized in that the substrate is selected from the group consisting of: substrates of chemical compounds capable of interacting with phosphorylated or nonphosphorylated vimentin, plastic multiwell assay plates, matrices for separating proteins according to their physicochemical characteristics, latex beads, and activated materials for specifically interacting with vimentin.

8. A method for identifying an anticancer agent that makes it possible to reduce the aggressiveness or invasiveness of a tumor by promoting vimentin phosphorylation, comprising:
culturing cells that produce nonphosphorylated vimentin;
bringing said cells into contact with at least one agent to be tested;
analyzing the vimentin produced in order to determine whether it is phosphorylated or nonphosphorylated after culturing of the cells in contact with the agent to be tested; and
selecting the at least one agent capable of inducing vimentin phosphorylation in the cancer cells tested.

9. The method as claimed in claim 8, characterized in that the cells that produce vimentin are cancer cells.

10. The method as claimed in claim 8, characterized in that the analysis of the vimentin produced is carried out by preparing a biological extract comprising vimentin and determining whether the vimentin thus extracted is phosphorylated or nonphosphorylated.

11. A method for treatment of cancerous tumors, comprising administering an agent that promotes vimentin phosphorylation in cancer cells to a subject.

12. The method as claimed in claim 10, wherein the analysis of the vimentin is carried out by a method selected from the group consisting of.
SELDI-TOF analysis,
the FACS method or equivalents thereof,
RIA isotopic labeling,
FRET fluorescence transfer,
the demonstration of immunocomplexes,
methods for detecting antigen-antibody reactions, and
electrophoretic migration tests with demonstration of posttranslational modifications of vimentin.
